(19) United States
(12) Patent Application Publication
(10) Pub. No.: US 2007/0049957 A1
(43) Pub. Date: Mar. 1, 2007

(54) CCUTTER

(76) Inventors: Pedro Benitez, Shrewsbury, MA (US); Rafael Garcia, Leon (MX)

Correspondence Address: Pedro Benitez, 6 Bannister Street, Shrewsbury, MA, 01545, US

(21) Appl. No.: 11/213,392

(22) Filed: Aug. 29, 2005

(51) Int. Cl. A61F 9/00

(52) U.S. Cl. 606/166

(57) ABSTRACT

The CCUTTER is an innovative ophthalmologic surgical instrument that greatly facilitates the performance of cataract surgery. Cataract surgery is the most frequently performed surgery in the United States, with over one million cataract surgeries done each year. A cataract is a clouding of the eye's internal lens, which interferes with the individual's ability to see clearly. During surgery, a continuous circular tear (called capsulorhexis) is made in the anterior capsule of the crystalline lens of the eye, and the front surface of the cataract is opened to allow access to the clouded tissue inside. The cloudy portion is then removed, leaving the thin clear back surface of the lens (called the posterior capsule) in place. A lens implant is then placed in the shell of the natural lens, and the incision is closed. The CCUTTER is designed to improve the predictability and consistency of performing the capsulorhexis during cataract surgery.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The CCUTTER is an innovative ophthalmologic surgical instrument that greatly facilitates and enhances the performance of cataract surgery. Cataract surgery is the most frequently performed surgery in the United States, with over one million cataract surgeries done each year. Cataract surgery has become one of the safest and most successful procedures in all of medicine. A cataract is a clouding of the eye's internal lens, which interferes with the individual's ability to see clearly. At present, there is no way to eliminate a cataract other than surgically removing it. Virtually everyone undergoing cataract surgery will have a plastic lens implant placed in the eye to replace the eye's natural lens. This lens implant will take over the focusing function of the eye's original lens. Cataract surgery is performed under a microscope to provide a magnified view of the eye. A small incision is made in the eye, and the front surface of the cataract is opened to allow access to the clouded tissue inside. The cloudy portion is then removed, leaving the thin clear back surface of the lens (called the posterior capsule) in place. This can be done either with a medical device called a phacoemulsifier or manually with extracapsular instruments (the CCUTTER is a new type of extracapsular instrument). The lens implant is then placed in the shell of the natural lens, and the incision is closed.

### BRIEF SUMMARY OF THE INVENTION

The CCUTTER is a new ophthalmologic surgical instrument for use in cataract surgery. The design of the CCUTTER dramatically improves predictability and consistency of capsulorhexis incisions performed by the freehand method. Capsulorhexis is defined as the making of a continuous circular tear in the anterior capsule during cataract surgery in order to allow expression or phacoemulsification of the nucleus of the lens.

### BRIEF DESCRIPTION OF THE SEVERAL VIEWS OF THE DRAWING

FIG. 1: CCUTTER cutting device

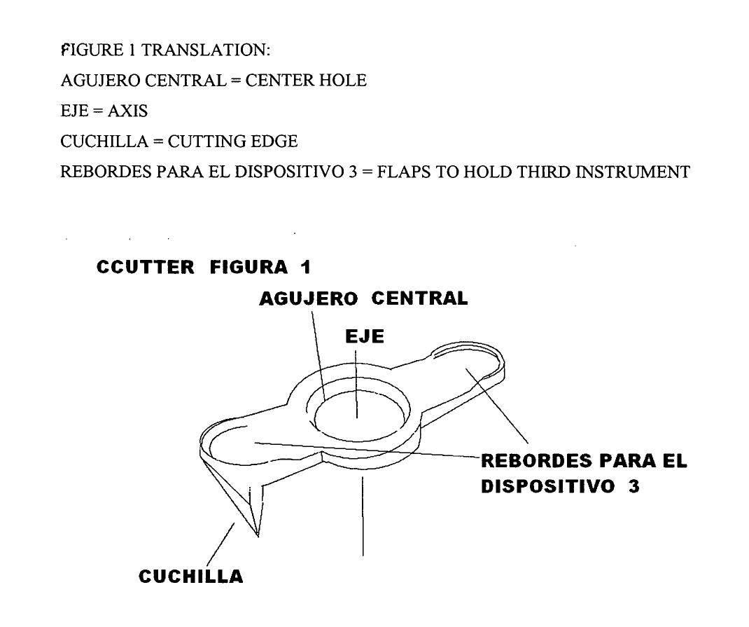

FIG. 2: Instrument for penetration, centering, and circular motion for cutting

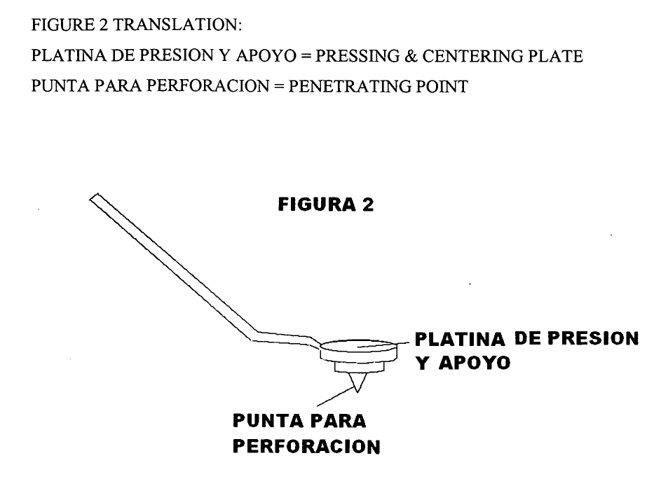

FIG. 3: CCUTTER rotator

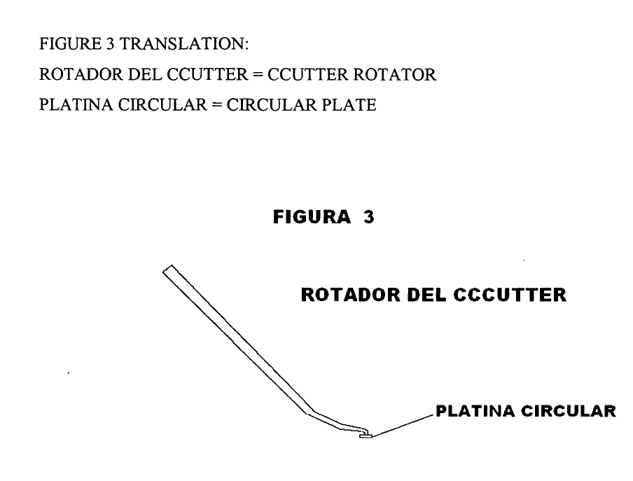

### DETAILED DESCRIPTION OF THE INVENTION

The CCUTTER is a device that is introduced in the anterior part of the eye during cataract surgery. This device permits the realization of a cut in the crystalline lens capsule using a rotary movement around a central axis. For this purpose, the device has a center hole, a cutting edge on one end, and an area to move the device on the other end.

Once the device has been positioned over the crystalline lens anterior capsule, it is centered and pressed with a second instrument. This second instrument has a handle that is angled at its distal part and has a termination with a major plate. The diameter of this major plate corresponds with the CCUTTER's center hole. A second plate has a diameter that allows adjustment to the CCUTTER hole by equalizing its thickness. The inferior portion of this second plate has a pointed zone with a conical form that has the function to penetrate the crystalline lens anterior capsule to serve as an axis to originate the circular movement of the CCUTTER.

Once the CCUTTER is positioned and the pressing & centering system is placed over the CCUTTER, a motioning force is applied to rotate the device. This rotation is achieved with the use of a third instrument that is placed in the rotation holes. This third instrument has an angled handle and a circular plate in its lower end that allows coupling with the CCUTTER.

1. We hereby claim that we have invented the CCUTTER, an innovative ophthalmologic surgical instrument to perform capsulorhexis during cataract surgery. Capsulorhexis is defined as the making of a continuous circular tear in the anterior capsule during cataract surgery in order to allow expression or phacoemulsification of the nucleus of the lens.
